(19) United States
(12) Patent Application Publication
(10) Pub. No.: US 2021/0346457 A1
(43) Pub. Date: Nov. 11, 2021

(54) COMPOSITIONS COMPRISING CANNABINOIDS AND PHYTOCHEMICALS, AND USES THEREOF

(71) Applicant: Fabrizio Di Stefano, Nobleton (CA)

(72) Inventor: Fabrizio Di Stefano, Nobleton (CA)

(21) Appl. No.: 17/307,320

(22) Filed: May 4, 2021

Related U.S. Application Data
(60) Provisional application No. 63/022,101, filed on May 8, 2020.

(51) Int. Cl. A61K 36/9068; A61K 36/32; A61K 36/9066; A61K 47/14; A61K 47/10; A61K 31/05; A61K 9/00; A61K 9/48

(52) U.S. Cl. CPC A61K 36/9068; A61K 36/32; A61K 36/9066; A61K 9/4825; A61K 47/10; A61K 31/05; A61K 9/0053; A61K 47/14

(57) ABSTRACT

The present application relates to compositions comprising cannabidiol and at least one phytochemical compound. The at least one phytochemical may be selected from Guggul extract (Commiphora mukul), turmeric extract (Curcuma longa), ginger extract (Zingiber officinale), Boswellia serrata extract, and combinations thereof. The composition may further be formulated in a lipid matrix. The present application further relates to uses and methods using the compositions of the application.

### REFERENCE TO RELATED APPLICATIONS

This application claims the benefit of U.S. Provisional Application No. 63/022,101 filed on May 8, 2020, which is incorporated by reference herein in its entirety.

### FIELD

The present application relates generally to formulations comprising cannabinoids and more specifically to formulations comprising CBD with other phytochemicals, preparations and uses thereof.

### BACKGROUND

Cannabis, commonly known as marijuana, is a product of the Cannabis sativa plant and the active compounds from this plant are collectedly referred to as cannabinoids. Marijuana leaves contain at least 70 cannabinoids. Among them, Δ9-tetrahydrocannabinol (THC), cannabidiol (CBD), and cannabinol (CBN) are the three major constituents. Cannabinoids have demonstrated several pharmacological effects on various diseases, disorders or conditions, including catalepsy, hypothermia, inflammation, pain, anxiety, insomnia, etc.

Despite their similar chemical structures, CBD and THC have very different psychoactive effects. THC is the primary psychoactive substance in marijuana, as CBD is a nonpsychoactive compound. In the therapeutic field, there is a great demand for THC free products, i.e. comprising CBD isolates, thus taking advantage of the therapeutic potentials of CBD without the psychoactive effect of THC. Typically, CBD isolate is a crystalline solid or powder that contains 99% pure CBD obtained from an extraction process to remove all the active compounds from the cannabis plant, followed by a refinement process that separate the other phytocannabinoids, including THC, and any plant matter, to isolate CBD chemical compound in its purest form.

Oral ingestion of CBD is popular because of its familiar nature, from eating food, drinking beverages, and swallowing daily vitamin. Unfortunately, while oral ingestion may be common, it's also the least effective method for absorbing CBD. The oral bioavailability of CBD is between 10% to 20%, and some studies even found it to be as low as 6%. Oral consumption of CBD forces it through the digestive system before reaching the bloodstream. Along the digestive track, a portion of the CBD is lost, discarded, or degraded, i.e. not absorbed. In addition to poor oral bioavailability, the consistent delivery of CBD molecules to the blood stream is also a challenge because of its rapid metabolism.

In view of the above, there is a need for CBD formulations which are orally ingestible and show good bioavailability, bioefficacy, and consistent delivery.

### SUMMARY

It is an object of the present application to provide formulations comprising CBD which are orally ingestible and show good bioavailability, bioefficacy, and consistent delivery.

It has been surprisingly shown herein that compositions comprising CBD combined with other phytochemicals provide good stability and bioavailability highlighting the surprising results obtained with the compositions of the application.

Accordingly, the present application includes compositions comprising a combination of CBD and at least one phytochemical compound.

In an embodiment, the phytochemical is selected from the group consisting of Guggul extract (Commiphora mukul), turmeric extract (Curcuma longa), ginger extract (Zingiber officinale), Boswellia serrata extract, and combinations thereof.

In an embodiment, the composition further comprises a lipid matrix. The lipid matrix may comprise a medium chain triglycerides and an emulsifier. In one embodiment, the emulsifier is glycerol monooleate, glycerol monostearate or any suitable nonionic surfactants, and combinations thereof.

In one embodiment, the composition is for oral administration.

The present application further provides a composition comprising cannabidiol, at least one phytochemical selected from the group consisting of Guggul extract (Commiphora mukul), turmeric extract (Curcuma longa), ginger extract (Zingiber officinale), Boswellia serrata extract, and combinations thereof, and a lipid matrix.

In an embodiment, the lipid matrix comprises a medium chain triglycerides and an emulsifier, and the emulsifier may be selected from the group consisting of glycerol monooleate, glycerol monostearate or any suitable nonionic surfactants, and combinations thereof.

In one embodiment, the composition is for oral administration.

The present application further includes a composition for use as an anti-inflammatory agent, an anti-nociceptive agent, an anti-cataleptic agent, an anti-epileptic agent, an anti-anxiety agent, an anti-insomnia agent, an immunity enhancer or a gastric disorders aid.

The present application further provides a method for preventing or treating inflammation, pain, catalepsy, epilepsy, anxiety, insomnia, or gastric disorders, or for enhancing immunity, said method comprising administering a therapeutically effective amount of a composition of the present application to a subject in need thereof.

The present application further includes a method for producing a composition comprising cannabidiol, at least one phytochemical selected from the group consisting of Guggul extract (Commiphora mukul), turmeric extract (Curcuma longa), ginger extract (Zingiber officinale), Boswellia serrata extract, and combinations thereof, and a lipid matrix, the method comprising:
a) mixing said cannabidiol, said at least one phytochemical and said lipid matrix;
b) formulating the composition in a pharmaceutically acceptable form.

In an embodiment, the pharmaceutically acceptable form is an oral formulation. In another embodiment, the oral formulation is a soft gelatin capsule.

In an embodiment, the lipid matrix comprises a medium chain triglycerides and an emulsifier. The emulsifier may be selected from the group consisting of glycerol monooleate, glycerol monostearate or any suitable nonionic surfactants, and combinations thereof.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

The term “composition(s) of the application” or “composition(s) of the present application” and the like as used herein refers to a composition, such a pharmaceutical composition, comprising combinations of compounds, as described in the application, and may further contain any acceptable carrier.

The term “and/or” as used herein means that the listed items are present, or used, individually or in combination. In effect, this term means that “at least one of” or “one or more” of the listed items is used or present.

As used in the present application, the singular forms “a”, “an” and “the” include plural references unless the content clearly dictates otherwise. For example, an embodiment including “a composition” should be understood to present certain aspects with one compound, or two or more additional compounds.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The term “suitable” as used herein means that the selection of the particular compound or conditions would depend on the specific manipulation to be performed, the identity of the compound to be transformed and/or the specific use for the compound, but the selection would be well within the skill of a person trained in the art.

The present description refers to a number of chemical terms and abbreviations used by those skilled in the art.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The term “subject” as used herein includes all members of the animal kingdom including mammals, and suitably refers to humans. Thus the methods and uses of the present application are applicable to both human therapy and veterinary applications.

The term “pharmaceutically acceptable” means compatible with the treatment of subjects.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. Treatment methods comprise administering to a subject a therapeutically effective amount of one or more of the compositions of the application and optionally consist of a single administration, or alternatively comprise a series of administrations.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition or manifesting a symptom associated with a disease, disorder or condition.

As used herein, the term “effective amount” or “therapeutically effective amount” means an amount of one or more compositions of the application that is effective, at dosages and for periods of time necessary to achieve the desired result.

The term “administered” as used herein means administration of a therapeutically effective amount of one or more compounds or compositions of the application to a cell, tissue, organ or subject.

### II. Compositions of the Application

It has been surprisingly shown herein that compositions comprising CBD combined with other phytochemicals provide good stability and bioavailability highlighting the surprising results obtained with the compositions of the application.

Accordingly, the present application includes compositions comprising a combination of CBD and at least one phytochemical compound.

For example, in Indian traditional medicine, guggulu has been used for thousands of years in the treatment of arthritis, inflammation, gout, rheumatism, obesity, and disorders of lipids metabolism. Guggulu is an extract from the plant Commiphora mukul (also known as Commiphora wightii) which contains diterpenoids, triterpenoids, steroids, long-chain aliphatic tetrols, aliphatic esters, ferulates, lignans, carbohydrates. Many steroid compounds have been isolated from guggul, including guggulsterone, and have been found responsible for many therapeutic benefits of this plant and extract.

Similarly, the turmeric plant (Curcuma longa), a member of the ginger family (Zingiberaceae), has long been used for its therapeutic properties. Turmeric extract comprises carbohydrates, water, proteins, fat, dietary minerals, essential oils, such as turmerone, germacrone, atlantone, and zingiberene, dietary fiber, and diarylheptanoids, a class including numerous curcuminoids, such as curcumin, demethoxycurcum in, and bisdemethoxycurcumin.

Another member of the same family is the Zingiber officinale, commonly called ginger. This plant comprises volatile oils, zingerone, shogaols, and gingerols with [6]-gingerol (1-[4′-hydroxy-3′-methoxyphenyl]-5-hydroxy-3-decanone) as the major compound. Fresh ginger also contains the enzyme zingibain which is a cysteine protease. Ginger is also commonly used to alleviate nausea and vomiting, and is suspected to have anti-inflammatory and pain relief effects.

A further example of phytochemical that may be used in the present application is extract from the Boswellia serrata plant. Some of its constituents include resin, essential oils, and polysaccharides, the resinous part comprising monoterpenes, diterpenes, triterpenes, triterpenic acids including various boswellic acid derivatives. The Boswellia serrata extract is also known to have various potential benefic effects such as pain relief and anti-inflammation.

It is submitted that phytochemical components, such as those of guggul turmeric, ginger and Boswellia serrata, may act synergistically with CBD to improve stability and bioavailability. Further, the phytochemical molecules present in those extracts may also act synergistically with CBD and make stable complexes in a lipid matrix. This may be useful for improving stability, and have a controlled release effect of the active ingredients.

In an embodiment, the compositions of the present application comprise cannabidiol and at least one phytochemical, which may be selected from the group consisting of Guggul extract (Commiphora mukul), turmeric extract (Curcuma longa), ginger extract (Zingiber officinale), Boswellia serrata extract, and combinations thereof.

These plant extracts may be commercially available, or may be extracted according to procedures known in the art.

In another embodiment, the compositions of the present application may further comprise a pharmaceutically acceptable carrier. It is to be understand that any suitable pharmaceutically acceptable carrier know in the art may be used.

In some embodiments, a composition of the application is orally administered, for example, including an inert diluent or an assimilable edible carrier, or it is enclosed in hard or soft shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers or diluents include lactose and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

In one embodiment, the pharmaceutically acceptable carrier is a lipid matrix. The lipid matrix may comprise triglycerides and an emulsifier. For example, the triglycerides are medium-chain triglycerides. In one embodiment, the emulsifier may be glycerol monooleate, glycerol monostearate or any suitable nonionic surfactants, and combinations thereof. It is to be understood that any lipid matrix, including suitable triglycerides and emulsifier, known in the art is contemplated.

The compositions of the application may be formulated to provide various dosages. For example, the CBD content in the formulations may be 10 mg, 20 mg, 30 mg, 40 mg, 50 mg, 100 mg, 250 mg, or up to 1000 mg. The dosage form, for example as a soft gelatin capsule, may be of different sizes, such as 500 mg, 600 mg, 750 mg or 1000 mg. Formulating specific dosage forms including the compositions of the present application is within the purview of a skilled person.

In some embodiments, the compositions of the application are administered to a subject by oral administration, but may be administered through inhalation, parenteral, buccal, sublingual, nasal, rectal, vaginal, patch, pump, topical or transdermal administration and the pharmaceutical compositions formulated accordingly. In some embodiments, administration is for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

### III. Methods and Uses of the Application

As CBD is suspected to have anti-inflammatory, analgesic, anti-nociceptive, anti-cataleptic, anti-epileptic, anti-anxiety, and anti-insomnia activities, or to act as an immunity enhancer or a gastric disorders aid, the compositions of the application may thus be useful for treating or preventing diseases, disorders or conditions associated with such effects. Therefore, the compositions of the present application may be useful as medicaments. Accordingly, the present application includes a composition of the application for use as a medicament.

The present application includes compositions for use as an anti-inflammatory agent, an analgesic agent, an anti-nociceptive agent, an anti-cataleptic agent, an anti-epileptic agent, an anti-anxiety agent, an anti-insomnia agent, an immunity enhancer or a gastric disorders aid.

The present application further provides methods for preventing or treating inflammation, pain, catalepsy, epilepsy, anxiety, insomnia, or gastric disorders, or for enhancing immunity, comprising administering a therapeutically effective amount of a composition of the present application to a subject in need thereof.

In an embodiment, effective amounts vary according to factors such as the disease state, age, sex and/or weight of the subject. In a further embodiment, the amount of a given composition that will correspond to an effective amount will vary depending upon factors, such as the given drug(s) or compound(s), the pharmaceutical formulation, the route of administration, the type of condition, disease or disorder, the identity of the subject being treated, and the like, but can nevertheless be routinely determined by one skilled in the art.

In an embodiment, the compositions of the application are administered at least once a week. However, in another embodiment, the compositions are administered to the subject from about one time per two weeks, three weeks or one month. In another embodiment, the compositions are administered about one time per week to about once daily. In another embodiment, the compositions are administered 2, 3, 4, 5 or 6 times daily. The length of the treatment period depends on a variety of factors, such as the severity of the disease, disorder or condition, the age of the subject, the concentration and/or the activity of the compositions of the application, and/or a combination thereof. It will also be appreciated that the effective dosage of the compositions used for the treatment may increase or decrease over the course of a particular treatment regime. Changes in dosage may result and become apparent by standard diagnostic assays known in the art. In some instances, chronic administration is required. For example, the compositions are administered to the subject in an amount and for duration sufficient to treat the subject.

In an embodiment, the subject is a mammal. In another embodiment, the subject is human.

### IV. Methods of Preparing the Compositions of the Application

Compositions of the present application can be prepared by various processes. The selection of a particular process to prepare a given composition is within the purview of the person of skill in the art.

In an embodiment, the compositions are prepared by mixing the components, and formulating the composition into an acceptable pharmaceutical form. A skilled person in the art would use methods know in the art for formulating the compositions into the desired final formulations. In one embodiment, the compositions are prepared and formulated as a soft gelatin capsule for oral administration.

### EXAMPLES

The following non-limiting examples are illustrative of the present application.

### General Methods

The starting materials used for the below formulations are commercially available, and were thus obtained from various commercial sources.

### Example 1—Formulations

This Example is directed to a concentration of CBD of 20 mg/dose, wherein the single oral dose is standardized to 750 mg in a soft gelatin capsule. The components include:
1. 100 mg—total guggul extracts (in which 2.5-5% are guggul sterones)
2. 50-100 mg curcuminoids
3. 4 mg Limonene
4. Lipid matrix—medium chain triglycerides (MCT) oil with glycerol monooleate.
5. 27-30 mg—cannabidiol

In a typical procedure, 50 g of curcuminoids—95% was added to 100 g of glycerol monooleate (GMO) at 55-60° C. along with constant stirring. This slurry was added to pre-warmed (55-60° C.). MCT oil—900 g, taken in a separate vessel with constant stirring. Stirring should continue for 45 min. To this colloidal solution, 25-30 g of turmeric extract (with 30% curcuminoids) added with constant stirring. Once this solution is homogenized, pre extracted guggul extract (2.5-5% guggul sterones) 130-140 g was added at 55-60° C., with constant stirring. Once the solution temperature came down to 30-35° C., 5 g of limonene was added and the entire solution was homogenized. To this phytochemical-lipid premix, 27-30 g of CBD isolate was added so that a 750 mg dose will have ˜20 mg CBD. After the addition, the solution was again homogenized, at a temperature 40-50° C. and soft gelatin capsules of 750 mg size were made.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein.

1. A composition comprising cannabidiol and at least one phytochemical compound.

2. The composition of claim 1, wherein said at least one phytochemical is selected from the group consisting of Guggul extract (Commiphora mukul), turmeric extract (Curcuma longa), ginger extract (Zingiber officinale), Boswellia serrata extract, and combinations thereof.

3. The composition of claim 1, further comprising a lipid matrix.

4. The composition of claim 3, wherein said lipid matrix comprises triglycerides and an emulsifier.

5. The composition of claim 4, wherein said triglycerides are medium-chain triglycerides.

6. The composition of claim 4, wherein said emulsifier is selected from the group consisting of glycerol monooleate, glycerol monostearate or any suitable nonionic surfactants, and combinations thereof.

7. The composition of claim 1, wherein the composition is for oral administration.

8. A composition comprising cannabidiol, at least one phytochemical selected from the group consisting of Guggul extract (Commiphora mukul), turmeric extract (Curcuma longa), ginger extract (Zingiber officinale), Boswellia serrata extract, and combinations thereof, and a lipid matrix.

9. The composition of claim 8, wherein said lipid matrix comprises triglycerides and an emulsifier.

10. The composition of claim 9, wherein said triglycerides are medium-chain triglycerides.

11. The composition of claim 9, wherein said emulsifier is selected from the group consisting of glycerol monooleate, glycerol monostearate or any suitable nonionic surfactants, and combinations thereof.

12. The composition of claim 1, wherein the composition is for oral administration.

13. A composition according to claim 1 for use as an anti-inflammatory agent, an analgesic agent, an anti-nociceptive agent, an anti-cataleptic agent, an anti-epileptic agent, an anti-anxiety agent, an anti-insomnia agent, an immunity enhancer or a gastric disorders aid.

14. A method for preventing or treating inflammation, pain, catalepsy, epilepsy, anxiety, insomnia, or gastric disorders, or for enhancing immunity, said method comprising administering a therapeutically effective amount of a composition of claim 1 to a subject in need thereof.

15. A method for producing a composition comprising cannabidiol, at least one phytochemical selected from the group consisting of Guggul extract (Commiphora mukul), turmeric extract (Curcuma longa), ginger extract (Zingiber officinale), Boswellia serrata extract, and combinations thereof, and a lipid matrix, the method comprising:
c) mixing said cannabidiol, said at least one phytochemical and said lipid matrix;
d) formulating the composition in a pharmaceutically acceptable form.

16. The method of claim 15, wherein said pharmaceutically acceptable form is an oral formulation.

17. The method of claim 16, wherein said oral formulation is a soft gelatin capsule.

18. The method of claim 15, wherein said lipid matrix comprises triglycerides and an emulsifier.

19. The method of claim 18, wherein said triglycerides are medium-chain triglycerides.

20. The method of claim 18, wherein said emulsifier is selected from the group consisting of glycerol monooleate, glycerol monostearate or any suitable nonionic surfactants, and combinations thereof.
